(19) United States
(12) Patent Application Publication
(10) Pub. No.: US 2023/0329967 A1
(43) Pub. Date: Oct. 19, 2023

(54) Foot Care Equipment

(71) Applicant: Mukesh Sharma, Surrey (CA)

(72) Inventor: Mukesh Sharma, Surrey (CA)

(21) Appl. No.: 17/905,988

(22) PCT Filed: Mar. 9, 2021
(86) PCT No.: PCT/IN2021/050222

§ 371 Date: Sep. 9, 2022

(30) Foreign Application Priority Data
Mar. 9, 2020 (IN) 202011010086

(51) Int. Cl. A61H 35/00; A61H 33/00; A61N 5/06; A61M 35/00; A61H 9/00; A61L 2/00; A61L 2/24; A61L 2/18; C02F 1/44; C02F 1/00

(52) U.S. Cl. CPC A61H 35/006; A61H 33/0091; A61H 33/6031; A61H 33/6047; A61N 5/0624; A61M 35/00; A61H 33/6089; A61H 33/0095; A61H 9/0071; A61L 2/0088; A61L 2/0047; A61L 2/24; A61L 2/18; C02F 1/44; C02F 1/008; A61H 2033/0037; A61H 2033/0012; A61N 2005/0661; A61N 2005/0642; A61N 2005/0654; A61H 2201/105; A61M 2210/08; A61H 2201/0207; A61H 2201/169; A61H 2033/0016; A61H 2201/025; A61H 2201/0214; A61L 2202/17; A61L 2202/15; A61L 2202/11; A61L 2202/14; A61L 2202/122; C02F 2209/02

(57) ABSTRACT

The new invention foot-care equipment unit is directly related to foot problems and cures from pain, numbness, stiffness, swelling, athlete's foot, blisters, bunios, corns, plantar fasciitis, heel spur, mallet or hammer toe, gout, ingrown toenail, nail hardening, fungal nail infection, plantar wart, stone bruise, morton neuroma, sesamoidits, poor blood circulation, crack heals, blood clots in the foot vain or excessive sweat, and or bad odours are common symptom of foot related issues or causes of poor foot health. The foot-care equipment a hands free solution for practicing random or regularly use of equipment for steaming, fermenting, massaging, cleaning and sanitizing, air blow drying, sterilizing and or scrubbing of foot or the feet's for cure or general health and hygiene as therapy or treatment. The foot-care equipment unit may comprise of semi-automatically functional in cleaning, sanitizing and sterilizing, and or drying of its equipment components for users.

The title of new invention is translated from Chinese JIAO HULI to English FOOT CARE, equipment unit comprising of different functional bodies into single foot-care unit, that has usage of air, water, in an equipment unit to obtain results.

The illustration of new invention described with some of the components already functional individually. Here this new invention use these individually functional equipment and parts of equipment body as assembly, assembled together for new invention, The component used such as air compressor, for air compression purpose, a water holding chamber and a chuck wheel run by electric motor for water rotation or circulation within chamber are all assembled along with. The water heating coil, and air heating coil with fan air blower attached along as assembly for different processes to attain different function results. Therefore the components and parts, of some component body, and individually functional units, are described in a listed here.
(1) New invention: Foot-care equipment unit.
(2) Air (purpose in use).
(3) Water (purpose in use).
(a) Air heater coil (electric heating metallic coil for hot air).
(b) Air blower fan (dry air blower fan).
(c) Air compressor (electric air compressor unit).
(d) Dispenser (ointments, oil's and powder dispenser).
(e) Water heating coil (submerged metallic water heating coil).
(f) Foot rest stand (foot placement metal stand).
(g) Spray gun (a spray gun, for water spraying).
(h) Electric motor and motor's (ac-dc electric power motors single & 3 phase).
(i) Rotary wheel disk (water rotation, wheel disk).
(j) Chamber (water storage chamber and units, components).
(k) Membrane (water filtration membrane, candle).
(l) Battery (dc power battery terminal).
(m) Drain valve (water drain valve outlet).
(n) PCB (printed circuit board power distribution switch).
(o) Wires (power supply conductors).
(p) Ultra violet lamp (sanitizing lamp).
(q) Switch or switch's (power, timer & other functional electric switches).
(r) Tubes (PVC, rubber pipes for air water connectivity).
(s) Body (metal, plastic or fiberglass material on base frame).
(t) Base frame (foundation structure angle frame base).
(u) Light switch (power indicators light switch).
(v) Equipment Skin (leather, PVC or other material unit cover).
(w) Water pump (waste water drainage, electric motor pump).
(x) Remote sensor (remote device control infrared sensor).
(y) Digital display temperature device (temperature display device).
(z) Adapter (ac/dc power reducer, converter).
(aa) Stone scrubber (telephonic pumice stone scrubber).
(bb) sensor device (target area sensors beams).

The foot-care unit a hands free service equipment comprise of listed components. Which is specified in the drawings and specification of descriptions in details. The foot care equipment unit is assembly of components by which foot or the feet's may rest in placed on footrest of the foot-care unit. The equipment's service involves hot, warm or cold water, hands free foot or feet's steaming, massaging, cleaning, sanitizing and blow drying and sterilizing with or without water.

The hot and cold water fermenting treatment of the foot or feet's with liquid solvents, hot or cold air blowing for hands free air drying of foot or feet's. Treating the foot or the feet's with Essential medically non-medically recommended oil's and powders.

Thereon the sterilize processes and methods describe further, in detail where the hands free medically recommended sanitization and sterilizing of foot or feet's with help of (p) ultra violet lamp (rays). The detail description involves the foot-care equipment unit and a hands free solution for the foot or the feet's. Where assembly includes listed components begins with the (t) base frame, which is metal frame, the (chassis) of the foot care equipment unit, may of any geometrical shape preferably rectangle, square and or hexagonal or combination of any of them includes wooden and or plastic or indulge with fabric material, to enhance equipment appearance or viability and functionality of the equipment unit. By mounting the (j) chamber unit on base frame, along with its components assembly together (as set of equipment unit). Turns the machine set into sustainable product of benefits, for human life includes health benefits and hygiene. The foundation frame is essential part to raise the equipment structure. The matter of facts and of all considering aspects of safety and security included, for the user while using the equipment or when in use.

The foot-care equipment unit accommodate clear functionality of different components attached to its body, this helps to drive the processes and methods with ease, of hands free usage of the equipment. The related components of the assembly of foot care equipment unit or units as required, by the component assembly to form (a single unit or multiple units combine together as chain). The (t) base frame metal of L shape considered as chassis, the structural foundation frame may be in a two piece structure fitted together as single pieces, rivet or bolted together. Which may include three to four set of hinges, to make two L shape inversely placed one on another and form J alphabet as shown in the drawings 1.

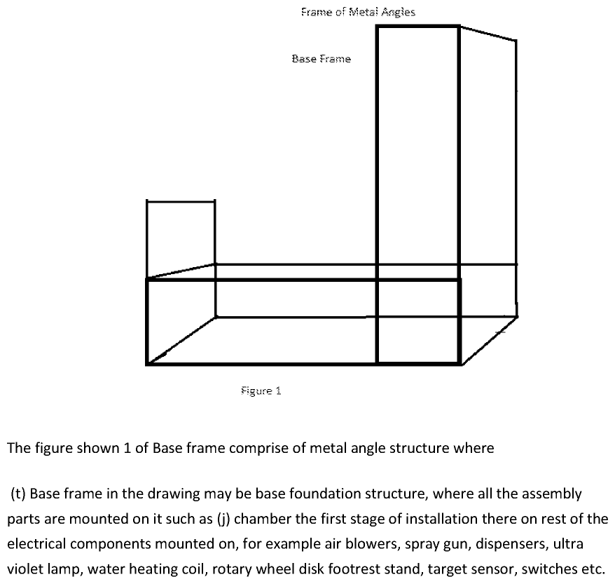

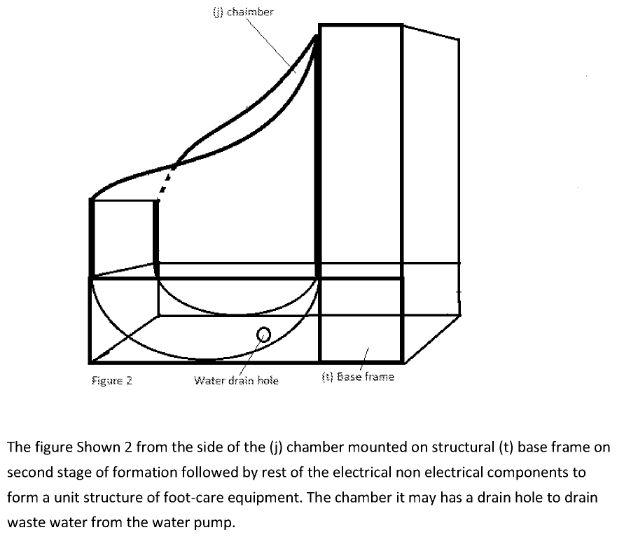

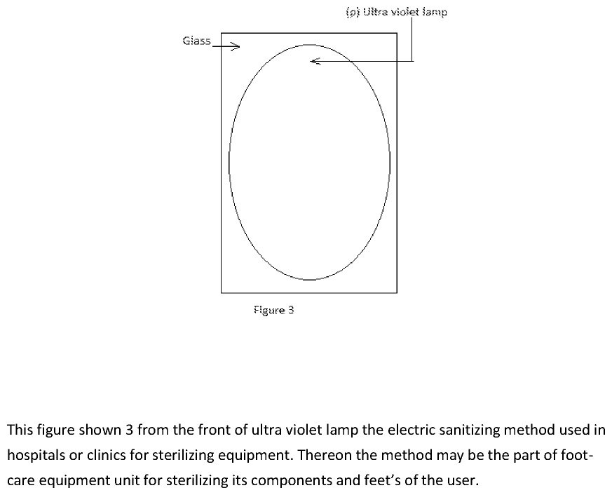

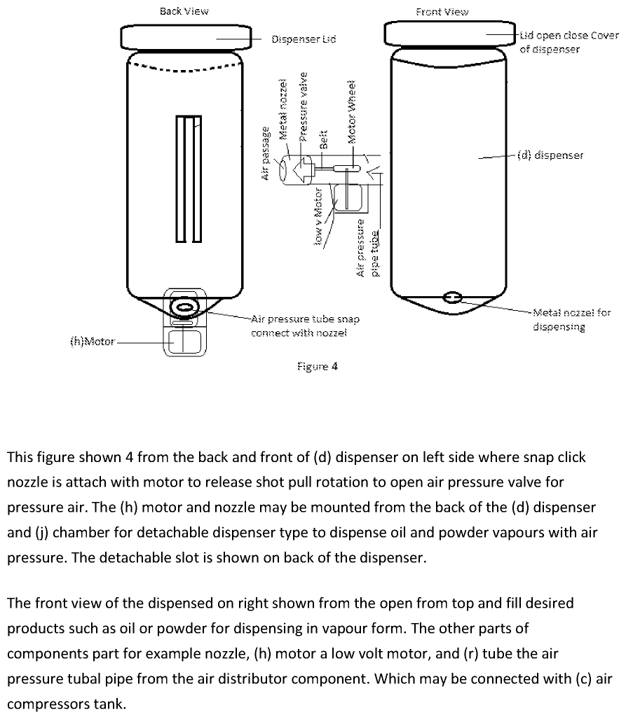

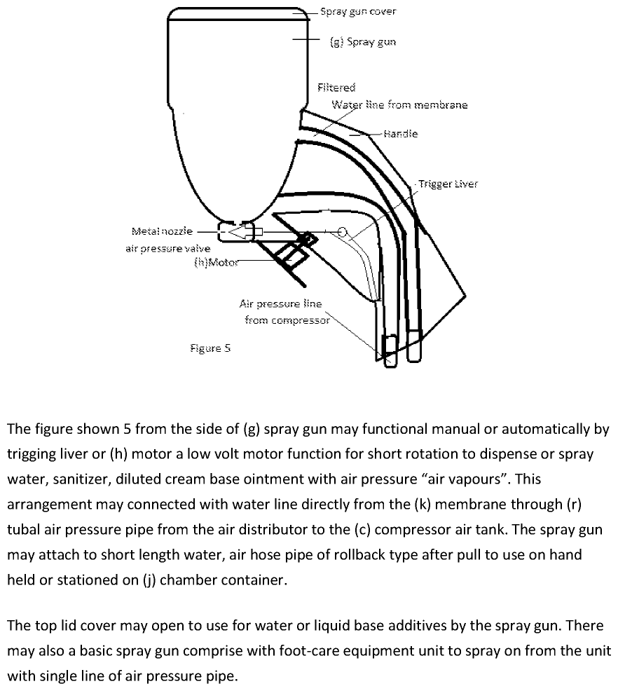

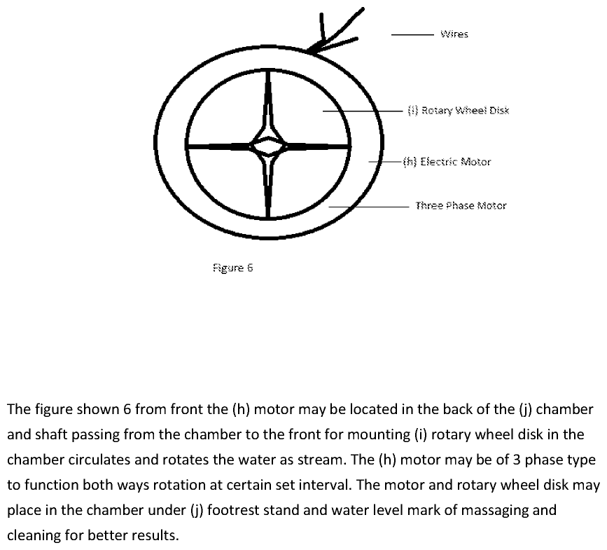

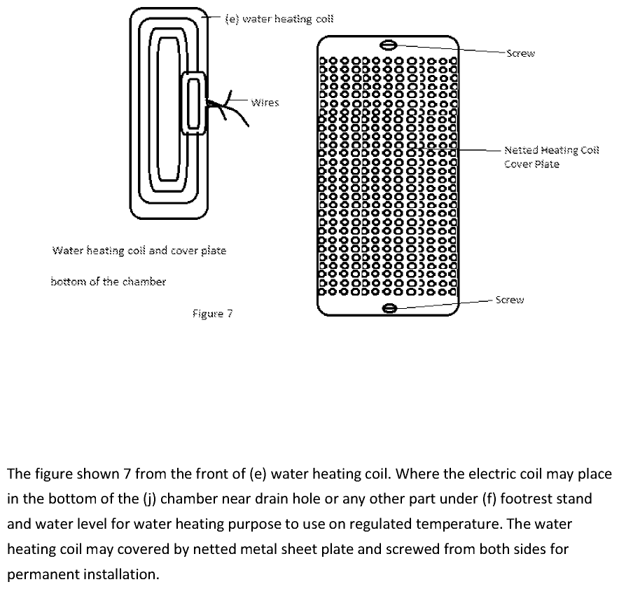

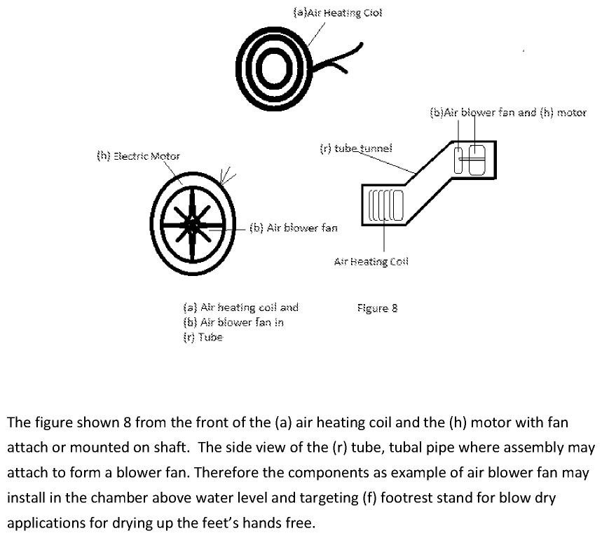

This arrangement may form one long J shape angle frame, permanently on the ground (unit assembly on base frame) stationed as base frame for the foot-care equipment unit. The base frame work may be of metal angles, for structure strength and to sustain all components, along with user weight, as safety factor. The foot care equipment's (chassis) base frame (t), the metal base frame has fitted two swingable folding cover doors, as part of base frame, functional from the front, this may be for safety reasons. The cover door's material of front part may be of any kind to cover the equipment from the front and up to the top. This compilation of foot-care equipment frame and door's has divided the frames in two part set, structural base frame and door's cover. The two swing doors when closed may allow to pass the legs and the feet's together from passage hole through close door from the bottom front part of the doors (as shown in the drawing FIG. 1).

Therefore the structure may designed with swing or convertible door cover for accessibility of foot-care equipment unit. The convertible or swing door's cover may be user friendly accessible to operate the foot-care equipment unit. The door cover material may of any kind from water proof PVC plastic, fiber-glass and shock proof and heat resistant material or materials. The unit cover may be open close door type to use the equipment from the front and user may stand vertically on foot-care equipment or may use on sitting position. Resting there feet's on the footrest stand when in use. The cover may lift from the top or the sides vice versa mechanically with (h) electric motor or manually to close or open the equipment unit from the front. The other specification are of foot-care equipment may comprise of, without any front cover. A single L shape foundation angle metal frame base laid to mount the component unit and remain open in all conditions.

Base Frame: Foot-Care Equipment Unit.

The(t) foundation of structural L shape metal base angle frame's dimensions may vary, example 1×1×1, 2×2×2 and 2×1×2 and 1×2×2 or 1×1×2 in ft. respectively. Length x Breath x Height. And all of different weight scale. The foundation frame work and or fitted components compiled together and mounted on (t) base frame of foot-care equipment unit. Includes assembly of listed parts of (a) to (bb) components and parts and one or two other such as air distributor switch. Some of the components descried are partly or fully hidden in the metal sheet body of the foot-care equipment unit. The components from the list ranges from, for example (a)(b)(c)(g)(h)(k)(l)(m)(n)(o)(p)(q)(r)(s)(t)(u)(w)(x)(y)(z)(aa)(bb) are the assembly compile fitting in and under (s) metal sheet body (casing) and slide down the unit to mount on (t) the base frame of the foot-care equipment unit further covered with (v) the equipment skin of PVC plastic or fiber glass or leather and or fabric, may include combination of any materials. The skin type may of electric shock resistant, heat resistant and water proof.

The mount assembly installed on base angle frame's metal plate, as (c) air compressor, (h) electric motor, and (j) chamber, the water chamber, (k) water filtration membrane, and (I) battery, these are the main component fitted on the bottom plate or of the angle frame according to their weight specification to balance the load factor of the foot-care equipment unit. The rest of the components (a) air heating coil, inside the tubal tunnel followed by (b) air blower fan for hot air blowing. The detachable (d) dispensers of oil, water and powder dispensers and (g) spray gun connected with (r) tube (the pipe) from the (c) air compressor for pressure air dispensing, of liquids or dry materials on the foot or the feet's, triggered by low volts small motors to release the air with pressure when triggered. The (I) battery of 12 v to 20 v dc may connected with described components through the battery terminals.

Thereon the water heating coil, air blower fan, rotary wheel disk motor, dispensers motor, and spray gun motor, ultraviolet lamps and all component may connected through PCB circuitry to the (q) switches the (power switch) and (o) wires the (insulated power supply conductors) to the battery terminal and (x) remote sensor connected and live wall slot plug switch. The foot-care equipment unit may be functional fully on power 110-220 volts AC/DC rechargeable battery or directly without battery terminal, on equipment specifications.

The components such as (a) air heating coil, air blower fan, (c) air compressor motor, (e) water heating coil, (h) electric motor, (n) printed circuit board (power distribution switch), (p) ultra violet lamps, (u) power light indicator switch, (w) water pump and (x) remote control sensor are connected via battery terminals and or direct to the wall plug switch.

Therefore the foot-care equipment unit may fully functional to the period of time with battery terminals in case of electric power failure or blackout.

### THE TECHNICAL PART

The method's and Process's describe in details as follows.

(t) Base Frame: Detail Description on Material

The base frame may comprise of solid material, selected for viability in term of equipment's strength, weight and safety factor, the most probable selection may of iron angels of ninety degree or may be of wood or plastic frame and or combination of these three to control vibrations.

(t) Base Frame: Detail Description on Weight

The base frame's weight may be lighter up to the load factor and the safety factor comprise (compromise). The base frame may strong enough to sustain equipment weight as well one and half of user's weight when use.

(t) Base Frames: Description on Size

The base frame's size as describe in feet's of Length x Breath x Height of any up to 2 cubic square feet's.

(t) Base Frames: Description on Shape

The base frames may comprise of materials to attain specific geometrical shape such as square and rectangle and or any feasible (working), user friendly model.

(t) Base Frames: Description on Bottom

The base frame's bottom part may be of wheel with position locks fittings and for movability and transportation from one place to another.

(t) Base Frames: Description on Top

The base frames top may covered with metal sheet, leather and or plastic, fiberglass or fabric accessory and or combination of all, this may enhance the equipment appearance.

(t) Base Frame: Sides

The base frames all and part of sides may be covered with sheet metal, hard PVC plastic or fiberglass or fabric and or with combination these indulging materials to prevent any threat to components circuits, other than that non-related objects pets and or its users from fall, injury, shock, burn, or any sort of damage as safety factor.

(a) Air Heating Coil: Process

The low volt air heating electric coil may be placed inside the metal or plastic tubal tunnel followed by (b) air blower fan and mounted on (j) the chamber and directed to target area on foot or the feet's placed on footrest stand. The air heating coil may turned on by (q) the switch when hot air is required to dry up the foot in winter with hot air, the process began when electric current passes through the coil resistance and air indulge with (contact) the hot coil and pushed by air blower fan to the target area of foot-care equipment unit. The process may continue by circuitry and the (q) switch wired through (n) PCB and the time bound timer switch from heating of coil to cut out process of the air heating coil or The air blower may connected together to (q) switches for three step's (high, medium and low) air heat control and fan speed control connected through (I) battery terminals and live wall slot plug switch.

(b) Air Blower Fan: Process

The air blower fan has air blowing functionality where fan is fitted in the tubal tunnel directed to the target area to blow dry the foot or the feet's surface. The one or two hot blower unit may be mounted on the (j) (chamber's) targeting footrest stand from the top, when foot drying process required by the user. The air blower fan may be connected to (q) switch for three step's air speed control from the fan. The blower fan complete circuitry completed with (I) battery terminals and live wall slot plug switch.

(c) Air Compressor: Process

The modified custom sized air compressor is the general air compressor (process) by which air may compressed through the motor and piston and filled and compress the air in a tank for later use. The compiled air compressor unit fitted in the back of the base unit and on base frame's metal sheet plate, completely hidden within the casing of the unit and under (j) chamber's metal sheet body covered from all the sides of the equipment unit. The compressor motor connected to the battery terminals wired and to the live wall plugs. Thereon compressor air tubal pipe may connected to one of the pipe from air distribution switch to the spray gun and to the oil and powder dispenser for series of processes.

(d) Dispenser's: Process

There are two type of dispenser's, one powder dispenser and second oil dispenser mounted on the (j) chamber of the unit from the front. These dispensers are may be of detachable type in the chamber's slot. The dispenser's has fine air (passing hole) nozzle from the bottom front of dispenser, targeting foot area for the air, water, powders and oil vapors applications. The (snap click) detachable connection of the dispenser's to the air tube allows the connectivity to the pressure air through (r) tubes of rubber. When triggered by the low voltage motors individually attach to each dispenser for short dispensing to release the product. The dispenser's motor circuit and connected through (o) wire to (n) PCB to (I) battery terminal, behind or back of the (j) chamber. The circuit complete with wired battery terminal and to the live wall slot switch. The dispenser's may be of any shape such as U cylindrical, square, and of or rectangular cube covered from the top by the lid cover. The user may open the lid and fill the choice of foot-care product, such as choice of oils and powder example talcum powder, medicated foot treating powder etc. The dispenser's body material may consist of from thin metal, PVC plastic and fiberglass and or combination of any may be use as feasible option. The dispenser's may be connected to low volt motor trigger (q) switch for programmed control dispensing, (of product) from the dispenser front to the user feet's.

(e) Water Heating Coil: Process

The water heating coil is similar to emersion tube or coil merged in water and +−terminals pass through the hole underneath (dry), the (j) chamber, connected with wires to battery terminals and to the wall plug. The (j) chamber (water chamber) fitted or mounted on (t) base frame angles. The water heating coil placed in a rectangular shape sink frame of the chamber at bottom of the chamber tank. And covered with fine holes netted sheet plate of metal, fitted with screws for permanent allocation of water heating coil or heating tube under (f) foot rest stand in the (j) chamber. Therefore the circuit through battery terminal and with (o) wires to the wall plug switch, render stability to use of equipment on either AC/DC power supply failure. The water heating coil may wired circuit through PCB and connected to (q) switch placed on the chamber's top. The temperature heat control switch may place on top of the equipment for heat control. This arrangement of switch regulates the temperature of the water heating coil to control the water temperature in the chamber. The (y) digital temperatures sensor device, display switch may place on top visible location of the chamber for safety reasons. The temperature detection metallic strip or needle is placed behind the chamber or middle of the (j) chamber under water level mark in the chamber for water temperature detection and wired to the display unit further (o) wired to the battery terminals to wall plug switch for circuit completion. The water heating coils process may cut or stopped manually by switch control or remote device.

(f) Footrest Stand: Method

The footrest stand may comprise of any metal, or cast metal, rigid heat resistant material insulation or alloy metal. The design may comprise of both feet's shape and or of any geometrical shape where foot or feet's accommodate easily for all foot size. This footrest stand may comprise of spikes, and or holes to circulate the water and air through and under the foot or the feet's for different foot applications such as steaming, massaging, cleaning, sanitizing, powder dusting and or oil spraying and sterilizing. The footrest stand may consist of movable seesaw type, where user may flexibly moves step up and down. While on sitting position from the front of the equipment and treat the foot or feet's from the bottom (surface) part. The footrest stand may design to lock the swing of footrest stand at fix position. The footrest stand may be located and placed in the (j) chamber under height of 5 inches of water level and the legs of the footrest passes through the chamber to the bottom of the base frame for stability. The footrest part may be detachable for cleaning purpose from the stand legs.

(g) Spray Gun: Process

The newly designed spray gun may consist of PVC plastic body or metal body with detachable lid or cover on the top and may attach to the low voltage motor for triggering process of air pressure dispensing through rubber tubal pipe and or (r) tubes of rubber from the (c) air compressor tank. The spray gun trigger may attach with low volt dedicated motor programed for short period rotation (o) wired to PCB control to the (I) battery terminal and further through the power reducer (z) adopter to the wall slot switch plug. The spray gun of foot-care equipment unit installed and placed in the center and front of the equipment of the (j) chamber above the water level mark. The spray gun may operate automatically as well manually from the trigger liver, connected through the rubber pipe to air compressor tank. The rubber pipe may of certain length and roll back type on spring roll, the pipe rollback to its position after use, such as when pull the spray gun for cleaning of the (j) chamber. The spray gun of the foot-care unit may be open from the top to add liquid product for use. The low volt electric motor may function as short trigger to release the air pressure from the spray gun for dispensing process. The air pressure line managed by triggering motor or manually functional gun liver to release air and water vapor (with diluted ointments, sanitizer and water vapor) to the target area. The other method where spray gun may directly connected through water line and air pressure line to the spray gun, where manual filling of water in the cup not required. The filtered water by membrane and through tubal pipe directly to the spray gun.

The water line may pass through (k) membrane (candle) water filtering membrane may of any shape and size installed behind or back of the (j) base frames plate. The water and air line separately through membrane, conduit under insulation along with electric (o) wires to the trigger motor. The electric power supply to the motor through circuits of switch, PCB, and sensor device, remote device to battery terminal and live wall slot plug switch.

(h) Electric Motor: Process

The electric motor or motors of different components motor of various power loads may be installed behind or back of the (j) chamber (completely hidden) for user's safety reason accordingly. Example air compressor, water spray gun motor, dispensers motors all are single phase motors, and water rotary wheel chuck motor which may be 3 phase for two way rotation. The electric motor bears different load capacity according to their functionality through (n) PCB connected (q) switch and through (o) wires the insulated power conductors to the (I) battery terminal+− and finally to the live wall slot plug switch. The motors may be categorized as consistent function motor such as, for (i) rotary wheel disk motor that run consistently through the power on/off (q) switch. The other low volt power functional motors may designed according to their process to run for short period of time or short rotation example dispenser motors and spray gun motors.

(i) Rotary Wheel Disk: Process

The rotary disk may rotates and circulates the water inside the (j) chamber through the three face motor for back and front motion rotation this may also be a single face motor for single direction rotation. The motor may located in the back of the chamber. The rotary wheel disk may be of metal disk plate or PVC plastic caped on motor shaft from the front of the chamber under water level mark. The rotary wheel disk rotation rotates the water inside the (j) chamber to massage and clean the foot or the feet's as and when required.

(j) Chamber: Process

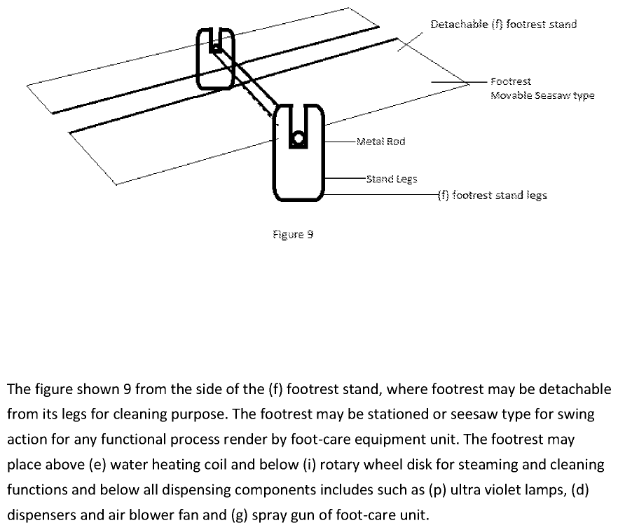

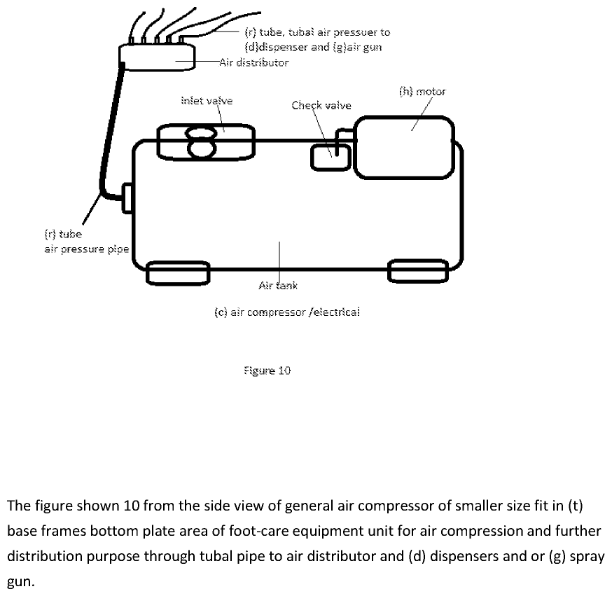

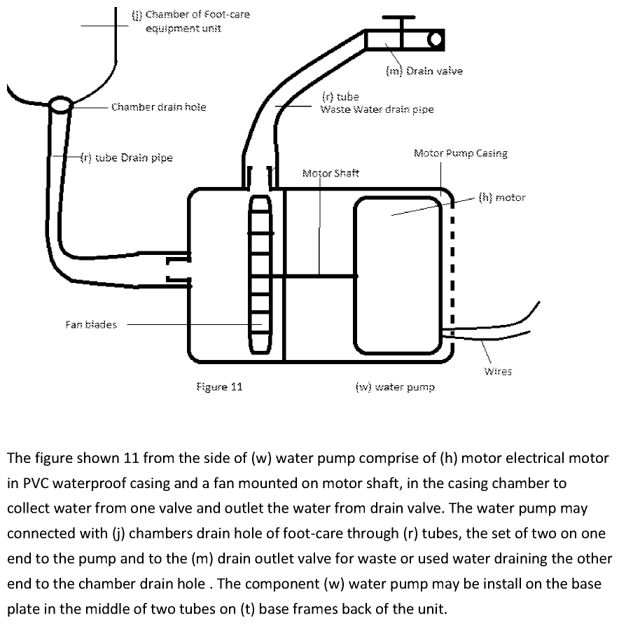

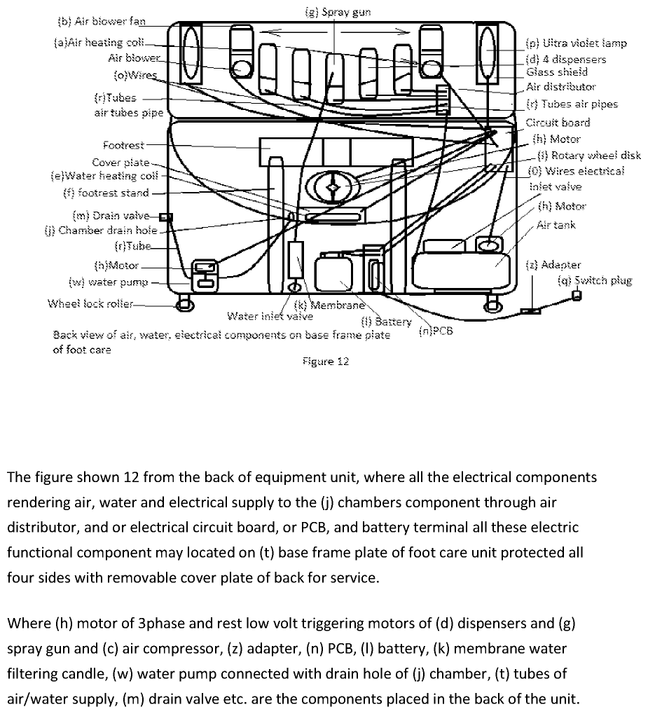

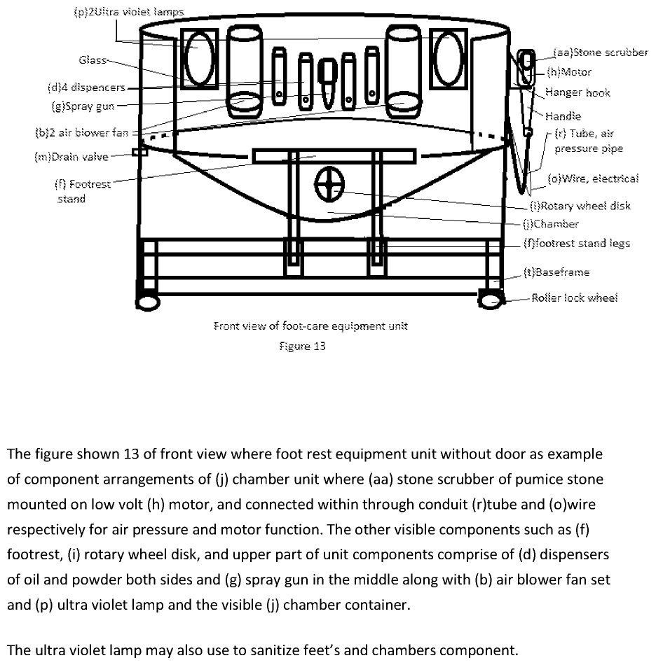

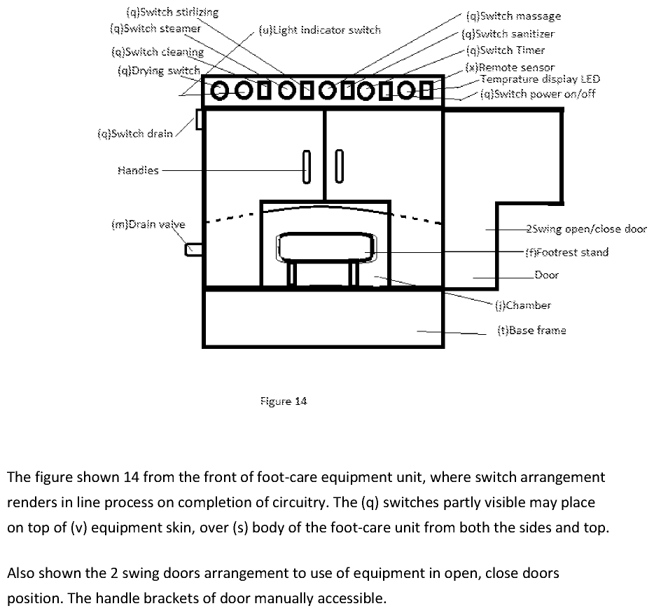

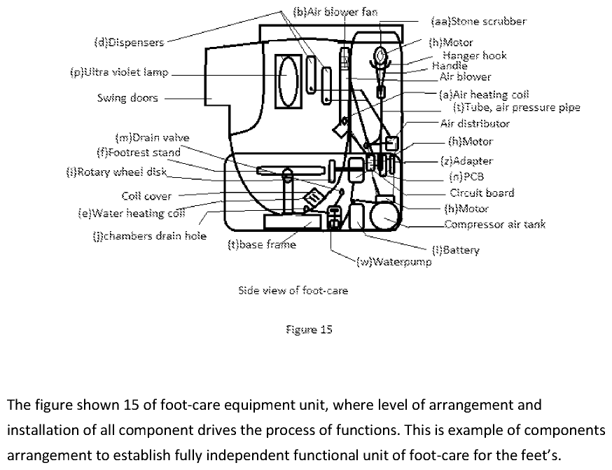

The chamber may consist of any metal or cast metal, thin sheet of alphabet letter J of higher leg type, covering the all the side as one piece container chamber as water sink open from the front, which may store the desired amount of water in it as per process requirement. And mounted on the (t) base frame angles from the front as shown in drawing FIG. 2. The chamber from the front hangered and with installed components such as (p) ultra violet sanitizing lamps, (d) oil, powder dispenser and (g) water spray gun and or (e) water heating coil, (b) hot/cold air blower fan and (f) footrest stand (i) rotary wheel disk. Further on top of that (q) switch or switches, (x) remote sensor, (u) light switch (power indicator), and target sensors, and (y) digital display temperature device. The (j) chamber may be covered from the front with open close swing door cover. The (j) chamber is used for resting of foot or feet's on footrest place and treat the foot or feet's by steaming, massaging, cleaning, drying, treating with oil and powder and or sanitizing and sterilizing process.

(k) Membrane: Process

The membrane candle may comprised of plastic, fabric, or metal body, and of carbon content inside of the candle to filter debris and dirt or other particles in the water. The membrane candle may be fitted on (t) base frame plate behind or back of the (j) chambers underneath and connected with the (r) tubes of rubber pipes (water hose) the water membrane may be installed in the middle of and between the one end to another of the foot-care equipment unit, feeds the clean water to the (g) spray gun cup as and when required from the wall tap for washing of equipment unit manually or cleaning and sanitizing of the feet's with water vapors.

(I) Battery: Process

The battery may comprise of lithium battery or new technology battery type. The battery may be installed in foot-care equipment unit under the (j) chamber and on (t) the base frames plate. By which the circuit completed to the all component and motors may feed the DC electric charge by the battery terminals through the (o) wired circuits. The other specifications of foot-care equipment unit may be used with or without the battery through the live wall slot plug switch directly. The battery may feed the power supply to all the components and motors in case of power failure or blackout.

(m) Drain valve: Process

The water drain valve outlet may comprise of PVC plastic, or metal valve on one side of the foot-care equipment unit as desired. The drain valve may be connected with (r) tubes the rubber tubal pipes to the (w) water pump install on base frame plate. And other end of tube pipe to the (j) chambers drain hole underneath bottom of the chamber near water heating coil for waste water drainage from the drain valve after use.

(n) PCB: Process

The PCB printed circuit board may be of any new design comprised of circuits for the low power programmed motors function to run the process of foot care equipment unit or units. The PCB board switch to run the (h) motors and other components in controlled manner, where lesser or more energy required by the motor or motors and components. The PCB allocates the energy supply to all and low voltage electric components and motors in function. Where the process involve to release of desired amount of powders, oil, water, air temperature or other applicable control elements of foot-care equipment. The PCB circuits may design in such a way to function all the components and motors of foot-care equipment unit with or without battery directly from the AC live wired wall slot plug switch.

(o) Wires: Method

The wires are the electric conductor may comprise of copper or aluminum of any gauge insulated, shock resistant circuits to all required electric components. The wires are of three type earth wire, neutral wire and power wire for complete circuits. The wires may laid as web of circuits wires through (I) battery terminal and or directly to the live wall slot plug switch includes and (z) adopter in a circuit chain.

(p) Ultra Violet Lamp: Process

The ultra violet lamp may be use for medically recommended ultraviolet rays on the foot or the feet's to sanitize and sterilize the targeted area of the feet's. The one or two ultra violet lamps may be installed in front part of the (j) chamber area right above the water level and right by the dispenser targeting all of container components and the foot or the feet's when in use to sanitize or sterilize. The ultra violet replaceable lamp may of any geometrical shape and size, attach or installed on holder or tubal strip through (q) switches and (o) Wires to the battery terminal and to the live wall slot plug switch.

(q) Switch or Switches: Process

The switch or switches are may comprise of PVC plastic as electric insulators, on position completes the (o) wired circuits of any functional model. Here switches are the functional model of foot-care equipment unit to start or stop any desired function when connected with wires to the battery terminal and live wall slot plug switch. Some functions may be time regulated timer switch or switches, of temperature adjustment of water, air in equipment. The switch process may complete as and when the (o) wires connected with switch and through (I) battery terminal or to the live wall slot plug switch.

(r) Tubes: Process

The tubes are hollow tunnel pipe may consist of rubber plastic or any kind of metal material. The tubes may be connected to the supply of any kind of liquid or air property through the tunnel. Furthermore the tube or tubes may connect one component to the other components in transferring material such as air or water. The tubes drive guided path to liquid and air from source to destination of foot-care equipment unit. The components connected to the tubes may be such as (w) water pump, (j) chamber and (g) spray gun (d) dispenser's etc. of foot-care equipment unit.

(s) Body: Method

The Body is the main body cover as (skin) of the equipment which covers the whole equipment's parts and its components and motors under. This body may be of any kind from metal, PVC plastic, fiber glass, and leather, and or fabric or combination of all for the foot-care equipment unit. There on some components or part of some components such as (q) switches, (y) digitally display temperature device, (u) light indicator (power switch), (x) remote sensor on top of the body (skin).

(t) Base Frame: Method

The base frame method describe in the description earlier on top at start point of the invention of foot-care equipment unit.

(u) Light Indicator Switch: Process

The light indicator switch may consist of lamp under plastic body may denote the power supply functionality to users of equipment circuit status for use when (o) wired and connected to the battery terminal and or to the wall slot plug switch.

(v) Equipment Skin: Method

Thereon to describe in detail description of the body skin is different from equipment skin. The body may be comprise of metal or any other material and equipment skin may comprise over body skin of leather, fabric, and or PVC Plastic or fiberglass, by covering skin type prevent shock and enhance the appearance of the equipment.

(w) Water Pump: Process

The water pump may comprise of metal and PVC plastic molded casing unit connected with (r) tubes of rubber or PVC flexi pipe and to the (j) chamber through pipe and further to the (m) drain valve to drain the water after use from the chamber of foot-care equipment unit. The water pump may be installed under the chamber and on the (t) base frame plate (base metal sheet) of the foot-care equipment unit. The water pump process start when connected through (o) wires and (q) switch circuits to the (I) battery terminal and or direct live wall slot plug switch.

(x) Remote Sensor: Process

The remote sensor device may be install on the equipment skin. The remote sensor may start and stop the process and functionality of the equipment's components and part of components. The motors for activation or stop of function such as power on off of the equipment furthermore motor or motors functionality of spraying or dispensing products from the components, temperature control of water in the chamber from the water heating coil, and or water pump process for draining water after use. The function start when remote device activates power supply through the sensor beam (infrared signals activates through remote device) to the (o) wires circuits connected with (I) battery terminal and or direct live wall slot plug switch.

(y) Digital Display Temperature Device

The digital display temperature device may be installed on the top, top front of (j) chamber and or on top of the equipment body skin. Therefore to display the current temperature of the water in the chamber of foot-care equipment unit. The digital display temperature devise may display once connected to (o) wires and through battery terminals to the live wall slot plug switch.
(z) Adapter: Process

An AC adapter, AC/DC adapter or AC/DC converter is a type of external power supply. The Use of an external power supply allows portability of equipment powered either by mains or battery without the added bulk of internal power components, and makes it unnecessary to produce equipment for use only with a specified power source. The adapter may be installed on the (t) base frame plate or on the wire chain of wall slot plug switch.

(aa) Stone Scrubber: Process

The Stone scrubber may comprise of pumice stone use for scrubbing of foot or the feet's. This stone may be of any shape and geometrical dimensions such as square rectangle, and round and flat. This stone scrubber may be fitted on hand held telephonic equipment where a low volt motor attach to the hand handle frame and install pumice stone scrubber on motor shaft. This telephonic hand held equipment is designed to move the pumice stone scrubber on linear motion or circular motion such as jig saw or grinder. The telephonic hand held stone scrubber may be installed on either side of the foot-care equipment's or within the (j) chamber's front. And furthermore telephonic handheld pumice stone scrubber may designed in such way that stone scrub the skin of the foot and air pressure line is provided to blow scraped dead skin from the foot when in use. Thereon the (r) tube (air pressure line) may laid along with electric (o) wire for electric power to low volt hand held motor for scrubbing stone and air supply from (c) air compressor to blowing dead skin from the feet's.

(bb) sensor device: Process

The sensor device process may compiled and use with all the step of processes where sensor devise beams the target such as user's feet on placed or removed from the chamber of foot-care unit equipment to start or stop any function processes powered by battery terminal or live wall slot plug switch through PCB and (q) switches and wires circuits.

Detail Description of Invention

The synopsis of description of foot-care equipment unit may as follows: The methods and process includes. Operational steps of functions as. The steaming: massaging: fermenting: cleaning: sanitizing: drying: sterilizing of the foot or the feet's on footrest stand in the unit's (j) chamber of the equipment unit.

The steaming: Step 1. This may be defined, at certain water level used for process. Example the hot or cold fermentation of the foot or the feet where medically recommended or aromatic oils mixed in water as treatment to the foot/feet's. A transparent cover or (base frame cover) as door of PVC plastic, where feet's may inserted from the bottom to use the foot-care equipment unit's for steaming process up to the desired period. The feet's may place on footrest. The water may be added up to the steaming (level) mark, mixed with recommended oils and on set temperature of use, the steaming of foot or feet's.

The massaging: Step 2. This may include up to the defined. At water level mark, the water may use for massaging the foot or the feet's with hot, and cold and or warm water, where water may be in stationed foot fermenting or massaging in rotational stream, through (i) rotary wheel disk within the (j) chamber of foot care equipment unit. The water may be added medicinal property (additives), and recommended oils and or water solvents homemade remedies. The foot or feet's may massage up to the desired period of time by the user. The massage process may stopped and control through remote device or the (q) switch.

The cleaning and sanitizing: Step 3-4. This may include up to the defined, at water level mark, of cleaning and sanitize of foot or the feet's. Where the water may be use for cleaning and sanitize of the foot or the feet's with hot, cold and or warm water, where water may be added in the (j) chamber container of foot care equipment unit and switch to function for rotation or circulation both the directions up to certain time period with (i) rotary wheel disk within the (j) chamber of foot care equipment unit. The water may be added soaps, liquid water solvents for cleaning and sanitize products. The foot or feet's may clean and sanitize in one or two steps first cleaning with recommended soap and liquid solvents and sanitizing with recommended products with or without water by the user. The process may be control through (q) switch or through remote device. The homemade remedies, treated water, aromatic water, Ayurveda's herbal or medicated medicine water, hygienic distilled, light emitted fused and sanitized water, may be used for foot treatment of foot care equipment unit(s).

The drying: Step 5. The step of process of foot or feet's drying may involve the hot, cold (b) air blower fan, where foot's may rest on footrest place and air blower fan may turn on with (q) switch or remote control device at certain interval or as desired by the user. The foot or feet's may be dried from the top and the bottom of the feet's while sitting on chair or a place and resting of the feet's on seesaw type (f) footrest stand in the (j) chamber of foot-care equipment unit.

The scrubbing: Step 6. The scrubbing process may include before or after drying of the foot or the feet's. The (aa) telephonic stone scrubber the hand held device may installed on either side of the equipment on the hanger hook. Where foot or the feet's may get scrubbed by holding the equipment's (component) and rubbed by pumice stone manually, or trigger low voltage power (h) motor installed on the hand held device and pumice stone mounted on the electric motor for scrubbing of the feet's and rotates in circular motion for scrubbing purpose. The scrubbing by (hand held) motor activates scrubbing process and blow down the dead skin from the user's foot with air pressure by the telephonic scrubber. The release of pressure air and motor rotation may function simultaneously by the liver when pressed on hand held telephonic device further connected with air tube from the (c) air compressor, (r) tubes from the air tank and wired with (o) wires circuit (n) PCB and (q) switches and (I) battery terminal and live wall slot switch plug.

The sterilizing: Step 7. The process step of disinfecting of foot or the feet's above all measured steps may involve sterilizing. The sterilizing method for the (user), foot or the feet's once placed on footrest stand and treated with ultra violet rays from (p) ultra violet lamp medically recommended and installed within the (j) chamber. The ultra violet lamp may covered with glass or transparent PVC plastic for shield. And the rays to pass on targeted areas of foot and equipment components in the chamber as well this process may be controlled by (q) switch or remote device up to the desired period of time. The sterilizing process includes the placement of the feet's in the chamber on footrest stand and ultra violet lamp may turn on. The ultra violet lamp may wired through the complete circuitry and connected with battery terminal and live wall slot plug switch and (n) PCB. The process may cut off by remote device or by (q) switch manually.

The oiling: Step 8. The step of process of oiling the foot or the feet's before or after other steps may set to different priority of applications. Where foot or the feet's may rest on foot-care equipment unit's (j) chamber and the massage oil's, treatment or homemade treated oils, and or aromatic oils may sprayed on the foots with dispenser cup filled from the top of the (d) dispenser, even medicinal anti-fungal, tubal cream base ointments may diluted with distal water in liquid form poured in dispenser and vaporable release by the dispenser and sprayed on the foot or the feet's through air pressure from the (c) air compressor (tank). This process may include user's choice of spraying oil through switch mode or by remote device of the foot-care equipment unit.

The dusting: Step 9. The step dusting of process of powder dusting on the feet's before or after other steps of process may set off, are different priority of applications. While sitting or standing on foot-care equipment unit conventional on the run. The user may rest foot or the feet's on footrest stand of unit's in the (j) chamber. And use the foot-care product in the powder form. The medically recommended and or general talcum powders may sprayed on the foots from the dispenser filled from the top of the (d) dispenser, even medicinal anti-fungal powders may dispense form the dispenser and sprayed on the foot or the feet's through air pressure release from the dispenser connected through the (c) air compressor (tank). This process may include user's choice of spraying of powder, through process mode set of the foot-care equipment or by remote device of the foot-care equipment unit ends the processes for user. Furthermore step 10 and 11 to be followed for maintenance of equipment. The foot care equipment has various functionality described under, in-relation to foot or feet's health. In term of foot infection, such as fungal infection, skin allergies of the foot or the feet's, athlete's foot, rashes and dryness of the foot-skin or cracks heals, excessive sweat and or bad odors from the foot or the feet's, improper blood circulation of the foot or the feet's, foot pain, dry foot nail's, beauty treatment of the foot or the feet's such as scrubbing and or skin treatment, vain clots therapy of the foots and or other medical non-medical foot's related complication's may be treated. The hands free foot-care processes, where foot or feet's may place on foot-rest stand of the equipment unit and steps of processes involve for therapy and treatment or in general foot care health.

The water darning: Step 10. After completion of the foot/feet's steps of processes the contaminated waste water may be drained through the (m) drain valve with (w) water pump function. Where the (r) tubal pipes connected from one end to the (j) chamber and other to the water pump same way one end to the water pump and other to the drain valve outlet The circuit may be wires to the pump motor to the switch, PCB, remote device sensor to the battery terminal and live wall slot plug switch.

The equipment cleaning and sanitizing: Process Step 11. The foot-care equipment unit after any process step, the series may be followed by in any order of step prior in function process which includes equipment's (j) chamber's cleaning with (g) spray gun installed in the middle of the chamber and (r) tubes connected with air distribution switch to the (c) air compressor (tank) for air pressure required through the air gun filled with water for vapor washing of the equipment with mild sanitizer liquid sprayed on the chamber's body. The rollback hose may attach to pull the spray gun 1m and spray water vapors on the chambers front body and roll back the pipe (r) back to its position after washing due to spring attach to the roller of hose pipe. Once washing and sanitizing is done. The foot-care equipment may set on sterilizing mode with (p) ultra violet lamps turned on with close door of the chamber for certain period of time as recommended. After sterilizing the foot-care equipment's sterilizing process completes, the equipment is ready to use again by the user.

Background: of Invention

The background of invention begins with foot health where foots are neglected generally and foot hygiene to maintain regular standards of practice. Keeping this mind the solution and preventive measures may require for people suffering from foot related problems such as athletic foot, fungal infections of the foot, nail hardening, poor blood circulation, crack heals, blood clots, sores and corn of the foot, bad smell or odors of the foot, swelling and or constant pain in the foot. This all with consideration of foot related issues and of remedy of the foot, the new invention drives in. Where the user may on standing or sitting position rest their feet's on footrest stand of foot-care equipment unit and hands free dry the feet's, oil them recommended oils, dust them with recommended powders, sanitize and sterilize them conventionally on the run. The foot-care equipment design or may design in such a way as user friendly for regular or random usage of the equipment enhance the good bold circulation, and cures the skin related (crack heals) and other infections and most importantly cures the decease as athlete's foot, fungal Infection, sores and dead skin and corns etc.

### SUMMARY OF THE INVENTION

The brief summary of the invention describes the essence of the invention concept. Generally for the foot related problems we all consult doctors' takes medication or the therapist for treatment. The summary include the invention of a compact appliance of foot-care equipment unit with all the features as treatment of foot or the feet's. This begins the newly designed equipment appliance which may comprise of multiple functionality in brief summary followed by steps.

The steaming: The process may be the first process of choice to begin with, where a small amount of water filled up to the steaming marked level for steaming the feet's for set amount of time. And dry the feet's with air blower without wiping out feet's may help in good blood circulation.

The massaging: Where the hot, warm, or cold massage treatment, may be provided to the feet's while on sitting or at standing position by filling the water in the chamber container to the marked level for water massaging, indulging with foot massage oils in the water for massage. The foot or the feet's placed in the appliance chamber's container from the front of the equipment, the two swing to close doors from the side to close the chamber. Therefore the process begins streaming of the water by the rotary wheel disk in the chamber and massage the feet's up to set interval. This may enhance foot health.

The cleaning and sanitizing: Thereon the new invention of cleaning and sanitizing of the foot or the feet's hands free. The water may be filled in the chamber container to the marked level of cleaning. The water may be hot, warm or cold as coil heating temperature set by user. The liquid foam of soap or shampoo may be added in the water. The foot or the feet's may place from the front of the foot-care equipment unit on foot rest stand for cleaning inside the chambers compartment. And two doors from the side close the chamber of the unit. The time of cleaning may set by user and the waste water may drained out from the equipment and the fresh water filled in with some sanitizer up to the level marked in the chamber container to sanitize the feet's resting on footrest on sitting or at standing

The drying: Therefore the process of functions over lapped of drying every now and then after any process. Where the foot need to be on the footrest stand in the equipment's chamber compartment for drying up feet's. The two air blowers fan set on blower dry up the feet's even from the top and the bottom part of the feet, as structural design of seesaw movable footrest stand. This process remain active with sensor device till the user with-draw the feet's from the foot-care equipment

The emulsification, oiling and powdering: Thereon the step may include emulsification tube, oils and or powder dusting. The medically recommended tube ointment (emulsification) may diluted with distil water for spraying on feet's same as powder dispensers dust powder from the dispenser connected with air pressure pipe from the air compressor tank of foot-care equipment unit. Therefore all these applications are hands free use by user and helps in control sweat and bad odors.

The sterilizing: The step may include sterilizing of the foot or the feet's with ultra violet rays the feet's. The sensors beam targeting the feet's allows the ultra violet lamp to stay on and sterilize the feet's. The sterilizing process may be at sitting or at standing position for set period of time by the foot-care equipment or may cut through remote device or by the user. The sterilizing of feet's enhance good health of feet's hygiene practice.

The stone scrubbing the stone scrubbing and removal of dead skin of the foot-heals or other parts of the foot or the feet's. Thereon a telephonic shape hand held scrubber installed on the foot-care equipment's body from the side of the unit hangered on the hook. The pumice stone roller of circular shape attach to a low volt motor along with air pressure pipe from the air compressor tank. The dead skin of the feet's may scrubbed by the pumice stone mounted on motor shaft of telephonic hand held unit component and blow down dead skin of the feet's by the air pressure. This may cure crack heals and foot appearance.

Equipment Cleaning to Sterilizing:

The equipment may designed in such a way it sterilize and clean chamber compartment semi-automatically by its user. The cleaning and sanitizing and sanitizing with ultra violet lamps of all chambers components including footrest stand. These following steps may completely make the equipment ready to use by the user.

1. A device for treating a person's foot, comprising:
a chamber configured to hold water at a specified water level during treatment;
a dispenser assembly disposed above a footrest, the dispenser assembly comprising:
a spray gun configured to dispense sanitizing liquid into the chamber for cleaning the foot, the spray gun mounted on an extendible hose line with a rollback mechanism operative to enable positioning of the spray gun within the chamber and the liquid provided via a liquid conduit, and
at least one dispenser configured to dispense footcare treatment substances into the chamber, the at least one dispenser including a nozzle targeting the foot during treatment; and
the footrest configured to support the foot in the chamber between the rotary disk and the nozzle during treatment,
a rotary disk disposed below the footrest in the chamber and configured to circulate water within the chamber to massage the foot during treatment; and
a water filtration membrane configured to filter the water in the chamber and to supply filtered water to the spray gun, the water filtration system including a candle filter for filtering particles from the water in the chamber.

2. (canceled)

3. The device of claim 1, further comprising: an air compressor coupled to the spray gun to supply the spray gun with pressurized air.

4. The device of claim 3, wherein the spray gun is configured to spray sanitizing liquid using the pressurized air to sanitize the chamber.

5. The device of claim 1, wherein the water in the chamber includes footcare treatment substances.

6. The device of claim 1, further comprising an ultraviolet lamp disposed above the footrest for sterilizing the foot during treatment and the chamber between treatments.

7. The device of claim 1, further comprising a plurality of ultraviolet lamps for sterilizing the chamber between treatments and for sterilizing the foot during treatment.

8. (canceled)

9. (canceled)

10. The device of claim 1, wherein the footrest is configured to simultaneously support two feet within the chamber.

11. The device of claim 10, wherein the footrest is supported by a metal rod defining a rotation axis of the footrest, the metal rod permitting a seesaw motion of the footrest about the rotation axis to allow movement of the feet in the chamber and the footrest is lockable on the rotation axis.

12. The device of claim 1, wherein the footrest comprises a plurality of apertures for facilitating circulation of water by the rotary disk through the footrest.

13. The device of claim 1, wherein the footrest is disposed at least five inches below the water level during foot treatment.

14. The device of claim 1, wherein the spray gun is coupled to a printed circuit board for automatic control of the spray gun.

15. The device of claim 1, wherein the spray gun includes a trigger for manual control.

16. The device of claim 1, further comprising: a hot/cold air blower disposed above the footrest for drying the foot during treatment and an air compressor to deliver air to the air blower.

17. The device of claim 1, further comprising: a heating coil disposed below the rotary disk for heating the water in the chamber during treatment.

18. The device of claim 1, wherein the dispenser assembly further comprises a powder dispenser and an oil dispenser attached to a conduit air line.

19. The device of claim 1, further comprising: a battery configured to operate at least one motor to circulate water in the chamber via the rotary disk and to operate the dispenser assembly.

20. The device of claim 1, wherein the dispenser assembly is configured to dispense sanitizing liquid for sanitizing the foot during treatment from an attachment to a conduit air line.

21. The device of claim 1, wherein the chamber includes a door positioned at a lateral side of the device above the water line to allow passage of the foot through the doors and prevent passage of water through the doors and enable a manual water feed from the front of the chamber.

22. The device of claim 1, wherein the nozzle is disposed above the footrest and the rotary disk is disposed below the footrest.

23. The device of claim 1, wherein the footrest is configured to support the foot in the chamber above the rotary disk and the dispenser assembly during treatment.

24. A method of operating a device for treating a person's foot, comprising:
receiving the foot in a chamber of the device;
supporting the foot on a footrest in the chamber;
heating water in the chamber based on a set temperature to steam the foot;
driving a rotary disk immersed in the water below the footrest to circulate the water in the chamber to massage the foot;
draining the water in the chamber;
after draining the water in the chamber, sterilizing the chamber, including applying ultraviolet radiation to the chamber using a plurality of ultraviolet lamps, and
spraying sanitizing liquid on the chamber using a spray gun disposed above the footrest.

25. The method of claim 24, further comprising:
using the plurality of ultraviolet lamps to generate ultraviolet radiation to sterilize the foot during treatment; and
dispensing sanitizing liquid above the footrest to sterilize the foot.

26. The method of claim 24, further comprising blowing air in the chamber to dry the foot.

27. The method of claim 24, wherein the driving a rotary disk immersed in the water below the footrest to circulate the water in the chamber to massage the foot includes circulating water through a plurality of apertures in the footrest.

28. The device of claim 1, wherein the specified water level is varied according to the foot treatment process being performed by the device.

29. The device of claim 1, further comprising a temperature control device for adjusting the temperature of the water within the device.

30. The device of claim 1, wherein the temperate control device is manually controlled by a user during treatment.

31. The device of claim 1, further comprising a pumice stone mounted on a motorized handle operated to move the pumice stone in either a liner motion or circular motion.

32. The device of claim 31, wherein the motorized handle further comprises a pressurized conduit air line operative to spray air onto the foot during operation of the pumice stone.
